(19) United States
(12) Patent Application Publication
(10) Pub. No.: US 2012/0111737 A1
(43) Pub. Date: May 10, 2012

(54) METHOD FOR PRODUCING CARBON MATERIALS HAVING NITROGEN MODIFICATION STARTING FROM CARBON NANOTUBES

(75) Inventors: Egbert Figgemeier, Leichlingen (DE); Elisabeth Zillner, Augsburg (DE); Benno Ulfik, Leverkusen (DE)

(73) Assignee: BAYER TECHNOLOGY SERVICES GMBH, Leverkusen (DE)

(21) Appl. No.: 13/263,861

(22) PCT Filed: Apr. 21, 2010
(86) PCT No.: PCT/EP10/02440

§ 371 Date: Jan. 17, 2012

(30) Foreign Application Priority Data
May 2, 2009 (DE) 10 2009 019 747.8

(51) Int. Cl. C01B 31/04; C25B 11/12; C07D 213/06; B01D 53/32; B82Y 99/00; B82Y 40/00

(52) U.S. Cl. 205/763; 546/251; 977/847; 977/902

(57) ABSTRACT

The invention relates to a novel process for producing carbon materials which are modified at least on their surface with pyridinic, pyrrolic and/or quaternary nitrogen groups starting out from carbon nanotubes.

The invention relates to a novel process for producing carbon materials which are modified at least on their surface with pyridinic, pyrrolic and/or quaternary nitrogen groups starting out from carbon nanotubes.

Carbon nanotubes have become generally known to those skilled in the art at least since their description in 1991 by Iijima (S. Iijima, Nature 354, 56-58, 1991). Since then, the term carbon nanotubes refers to cylindrical bodies comprising carbon and having a diameter in the range from 3 to 80 nm and a length which is a number of times, at least 10 times, the diameter.

A further characteristic of these carbon nanotubes is layers of ordered carbon atoms. Synonyms for carbon nanotubes are, for example, “carbon fibrils” or “hollow carbon fibres” or “carbon bamboos” or (in the case of wound structures) “nanoscrolls” or “nanorolls”.

Owing to their dimensions and their particular properties, these carbon nanotubes are industrially important for the production of composite materials. Further important possibilities are in electronics and energy applications since they generally have a higher specific conductivity than graphitic carbon, e.g. in the form of conductive carbon black. The use of carbon nanotubes is particularly advantageous when these are very uniform in respect of the abovementioned properties (diameter, length, etc).

The possibility of doping these carbon nanotubes with heteroatoms, e.g. atoms of the fifth main group (e.g. nitrogen) during the process for producing the carbon nanotubes in order to obtain basic catalysts is likewise known.

The generally known methods of producing nitrogen-doped carbon nanotubes are based on the conventional production methods for classical carbon nanotubes, for example electric arc, laser ablation and catalytic processes.

Electric arc and laser ablation processes are characterized, inter alia, in that carbon black, amorphous carbon and fibres having large diameters are formed as by-products in these production processes, so that the resulting carbon nanotubes usually have to be subjected to complicated after-treatment steps, which makes the products obtained from these processes and thus these processes economically unattractive.

In contrast, catalytic processes offer advantages for economical production of carbon nanotubes since a product having a high quality may be able to be produced in good yield by these processes. In the case of the catalytic processes, a distinction is usually made between processes using supported systems and “floating catalyst” processes.

The former usually comprise catalysts located on a support matrix which may itself be active, while the latter term usually refers to processes in which the catalyst is formed from a precursor under the reaction conditions for producing the carbon nanotubes.

Maldonado et. al. (Carbon 2006, 44(8), 1429-1437) disclose a typical embodiment of the “floating catalyst” processes according to the prior art. The process for producing nitrogen-doped carbon nanotubes is characterized by the in-situ decomposition of a catalytic component (ferrocene) in the presence of NH₃ and xylene or pyridine. A general disadvantage of such processes is that the use of expensive organometallic chemicals for carrying out such processes is unavoidable. Furthermore, a majority of organometallic chemical compounds are highly hazardous to health or are at least suspected of being carcinogenic.

WO 2005/035841 A2 discloses a process which comprises the production of electrodes comprising a conductive core and a layer of nitrogen-doped carbon nanotubes deposited thereon. The process is a “floating catalyst” process according to the above definition and has the associated disadvantages.

Van Dommele et al. and Matter et al. (S. van Dommele et al., Stud. Surf. Sci. and Cat., 2006, 162, 29-36, ed.: E. M. Gaigneaux et al.; P. H. Matter et al., J. Mol. Cat A: Chemical 264 (2007), 73-81) each disclose a typical embodiment of supported processes according to the prior art, using nitrogen-doped carbon nanotubes on a supported catalyst comprising cobalt, iron or nickel on an SiO₂ or MgO matrix in order to deposit acetonitrile or pyridine as carbon and nitrogen source thereon in the form of carbon nanotubes. These production processes are characterized, inter alia, in that they are carried out in fixed-bed reactors in the laboratory.

An alternative to these processes is disclosed in US 2007/0157348, in which nitrogen-doped carbon nanotubes are produced catalytically in a fixed bed using an H₂O plasma. The process comprises, inter alia, production of a catalytic metal layer on a substrate surface on which the carbon nanotubes are subsequently formed. This is accordingly a particular embodiment of the supported process variant for producing carbon nanotubes.

The methods just described (supported and “floating catalyst” processes) are also generally known to those skilled in the art under the collective term of catalytic, chemical gas phase deposition (catalytic chemical vapour deposition; CCVD). A characteristic of all CCVD processes is that the metal component which is used and is referred to as catalyst is consumed during the course of the synthesis process. This consumption is generally attributable to deactivation of the metal component, e.g. due to the deposition of carbon on the total particle, which leads to complete covering of the particle (this is known as “encapping” to those skilled in the art).

Reactivation is generally not possible or not economically feasible.

Processes such as, for example, the previously disclosed processes of Van Dommele et al. and Matter et al. and also of US 2007/0157348 are not advantageous since they are carried out in fixed-bed reactors and exchange and replacement of any deactivated catalyst is thus possible only with great difficulty.

Such supported embodiments are likewise disadvantageous in that the catalytic metal sites available for the reaction of starting material to form nitrogen-modified carbon nanotubes are present only on the surface of the particles or of the substrate. This in turn leads to an inherent limitation of these embodiments in respect of the achievable yield of nitrogen-modified carbon nanotubes per particle or amount of substrate. Furthermore, the types of reactor used are not suitable for long-term continuous operation because of the large change in volume of the fixed bed during nitrogen-modified carbon nanotube formation. Scale-up of these types of reactor is therefore not possible in an economical fashion.

Processes which are not subject to this restriction are, in particular, fluidized-bed processes.

DE 10 2006 017 695 A1 discloses a process which comprises the production of carbon nanotubes in a fluidized bed. In particular, an advantageous mode of operation of the fluidized bed by means of which carbon nanotubes can be produced continuously with introduction of fresh catalyst and discharge of product is disclosed. It is likewise disclosed that the starting materials used can comprise heteroatoms.

On the other hand, subsequent modification of the carbon nanotubes with nitrogen is not disclosed.

An alternative for achieving high yields based on the catalyst used and for achieving advantageous product properties of carbon nanotubes is disclosed in DE 10 2006 007 147. Here, a catalyst which comprises a high proportion of catalytically active metal components and therefore leads to the desired high yields is disclosed. It is likewise disclosed that the starting materials used can comprise heteroatoms.

On the other hand, subsequent modification of the carbon nanotubes with nitrogen is not disclosed.

The as yet unpublished German Patent Application No. DE 10 2007 062 421.4 discloses an advantageous process by means of which the abovementioned disadvantages of the prior art can be overcome, but the modification with nitrogen is still disclosed only in the actual process for producing the nitrogen-modified carbon nanotubes by use of at least one starting material comprising carbon and/or nitrogen.

Thus, DE 10 2007 062 421.4 does not disclose a process for the subsequent modification of carbon nanotubes with nitrogen in order to produce carbon materials modified on the surface with pyridinic, pyrrolic and/or quaternary nitrogen groups.

A process for the subsequent modification of carbon nanotubes with amine nitrogen is disclosed by Z. Konya et al. in “Large scale production of short functionalized carbon nanotubes” (Chem. Phys. Letters, 360, 2002: 429-435).

Z. Konya et al. disclose that milling carbon nanotubes in the presence of reactive gases such as hydrogen sulphide, ammonia, chlorine, carbon monoxide, methyl mercaptan and phosgene makes it possible to obtain carbon materials which are modified with chemical groups originating from these reactive gases. Thus, it is disclosed that in the case of milling with ammonia amine groups and/or amide groups can be bound to the carbon nanotubes. However, it is further disclosed that the ball mill used is either flushed with nitrogen or baked under reduced pressure before the reactive milling and the reactive gas is then introduced while milling. After the reactive milling, the remaining reactive gas is removed again either by flushing with nitrogen or under reduced pressure.

Z. Konya et al. also do not disclose that the process starting out from carbon nanotubes makes it possible to obtain carbon materials which comprise pyridinic, pyrrolic and/or quaternary nitrogen groups at least on their surface. In addition, the process of Z. Konya et al. has the disadvantage that the gas ammonia, which is toxic and corrosive compared to nitrogen, has to be used to obtain the amine nitrogen groups on the surface of the carbon nanotubes.

Proceeding from the prior art, there is thus still the problem of providing a process which allows graphitic carbon materials and in particular carbon nanotubes which comprise pyridinic, pyrrolic and/or quaternary nitrogen groups at least on their surface to be obtained and in which the modification with nitrogen can be carried out outside the process for producing the carbon nanotubes and the modification with nitrogen is carried out very simply and inexpensively under conditions which are unproblematical in process engineering terms.

We have now surprisingly found, as first subject of the present invention, that a process for producing graphitic carbon materials comprising pyridinic, pyrrolic and/or quaternary nitrogen groups at least on their surface starting out from carbon nanotubes, characterized in that carbon nanotubes are milled under a nitrogen atmosphere, is able to solve this problem.

For the purposes of the present invention, graphitic carbon materials comprising pyridinic, pyrrolic and/or quaternary nitrogen groups at least on their surface are carbon materials which comprise graphitic carbon and on their surface have a proportion of at least 1 atom % of nitrogen in pyridinic, pyrrolic and/or quaternary form.

In the preferred embodiments of the process of the invention described below, graphitic carbon materials which comprise pyridinic, pyrrolic and/or quaternary nitrogen groups at least on their surface have at least 5 atom % of nitrogen in pyridinic, pyrrolic and/or quaternary form can also be obtained.

For the purposes of the present invention, the surface of the graphitic carbon materials is the proportion of the graphitic carbon materials comprising pyridinic, pyrrolic and/or quaternary nitrogen groups at least on their surface which is accessible to measurement by means of X-ray photoelectron spectroscopy (hereinafter also referred to XPS for short). A person skilled in the art would generally know that X-ray photoelectron spectroscopy (XPS) allows analysis of the material to be examined only to a particular depth which depends on the analytical instrument and on the sample examined.

Preferred graphitic carbon materials comprising pyridinic, pyrrolic and/or quaternary nitrogen groups at least on their surface are carbon nanotubes comprising pyridinic, pyrrolic and/or quaternary nitrogen groups at least on their surface.

These can, according to the present invention, likewise have a proportion of at least 1 atom % of nitrogen, preferably at least 5 atom % of nitrogen, in pyridinic, pyrrolic and/or quaternary form on their surface after the process disclosed here.

The graphitic carbon materials obtained by the process of the invention and its preferred embodiments can have this proportion of nitrogen not only in the abovementioned form as pyridinic, pyrrolic and/or quaternary nitrogen groups but also in further forms. An example of such a further form is amine nitrogen.

However, it is essential to the present invention that pyridinic, pyrrolic and/or quaternary nitrogen groups can be obtained on/in the graphitic carbon materials by means of the process of the invention, which has in the previously known prior art either not been possible or possible only in the course of the production process of nitrogen-modified carbon nanotubes.

The process of the invention is particularly advantageous because, firstly, it allows subsequent modification of carbon nanotubes with the abovementioned forms of nitrogen at least on their surface, secondly is particularly simple to carry out compared to the catalytic reaction processes and, finally, simultaneously allows any agglomerates of carbon nanotubes present at the beginning to be destroyed so that finely dispersed carbon nanotubes having pyridinic, pyrrolic and/or quaternary nitrogen groups at least on their surface can be obtained. Furthermore, the use of toxic and corrosive gases such as ammonia can be dispensed with.

During studies on breaking up aforementioned agglomerates, it has surprisingly been found that chemical modification of the carbon nanotubes with the various forms of nitrogen occurs even when the carbon nanotubes are stressed by milling under a nitrogen atmosphere so as to give graphitic carbon materials having pyridinic, pyrrolic and/or quaternary nitrogen groups. In contrast to the opinion prevailing in the prior art (cf. Z. Konya et al.), nitrogen is therefore not an inert gas but can be used directly for producing graphitic carbon materials having pyridinic, pyrrolic and/or quaternary nitrogen groups.

The nitrogen atmosphere present in the process of the invention thus means that the milling according to the invention takes place in an environment in which the proportion of nitrogen is at least 78% by volume, preferably at least 90% by volume.

In particularly preferred embodiments of the process of the invention, the proportion of nitrogen in the environment is at least 99% by volume. However, the process of the invention can also be carried out under ambient air.

The advantages of the resulting graphitic carbon materials having pyridinic, pyrrolic and/or quaternary nitrogen groups are based on, in particular, the fact that they are, firstly, catalytically active as has already been emphasized in, for example, DE 10 2007 062 421.4 and, secondly, further chemical groups can be bound to such nitrogen groups so that the resulting graphitic carbon materials having pyridinic, pyrrolic and/or quaternary nitrogen groups can be chemically functionalized in a particularly advantageous way, which has likewise been indicated in DE 10 2007 062 421.4.

The milling according to the invention can be carried out in all milling apparatuses which are generally known to those skilled in the art and are adapted to the fact that the material being milled can have the dimensions of carbon nanotubes. The above adaptation can easily be carried out by a person skilled in the art.

In a preferred embodiment of the present invention, milling is carried out in a milling media mill.

Nonexhaustive examples of such milling media mills are planetary mills, ball mills, vibratory mills and stirred ball mills. Preferred milling media mills are planetary mills.

The use of a milling media mill is particularly advantageous because not only does frictional stress on the carbon nanotubes take place in such apparatuses but strong impulse forces of milling media and/or wall of the milling chamber also occur due to impingement of milling media against milling media and milling media against the wall of the milling chamber, so that when one or more carbon nanotubes are present between milling medium and milling medium, or between milling medium and the wall of the milling chamber they are subjected to intensive mechanical stress.

Without wishing to be tied to a theory, it appears that the brief, sufficient mechanical stressing under a nitrogen atmosphere is sufficient to make at least the surface of the carbon nanotubes accessible to chemical bonding of the nitrogen present, so that by this means alone it is possible to produce the graphitic carbon materials comprising pyridinic, pyrrolic and/or quaternary nitrogen groups at least on their surface.

The milling in the process of the invention can be carried out for a period of from one minute to sixteen hours. Preference is given to carrying out milling for a period of from four to eight hours.

The lower limit to the period for which milling is carried out is based on the fact that the probability of a carbon nanotube to be milled being subjected to stress once is determined by the time.

It has been found that a period of even one minute leads to measurable modification of the carbon nanotubes with nitrogen, so that from this time onwards a sufficient probability of a stress being applied once can be assumed.

The upper limit to the period of time is advantageous because continuing stressing of the carbon nanotubes during milling also increases the probability that these will be converted into an amorphous state of carbon at least on the surface. However, since, in particular, the structure of the carbon nanotubes and also that of the graphitic carbon materials comprising pyridinic, pyrrolic and/or quaternary nitrogen groups at least on their surface is important for their positive properties, this should be avoided. It has been found that this can appropriately be achieved by limiting the milling time to a maximum of 16 hours.

It is also clear from this that, depending on the milling time, the carbon nanotubes can be comminuted by the milling in such a way that they no longer come under the general abovementioned definition of carbon nanotubes as is at present generally recognized in the art, since the relevant diameter to length ratios are no longer fulfilled. However, since carbon nanotubes generally comprise a graphitic arrangement of the carbon in short-range chemical order, for the purposes of the present invention reference will be made to graphitic carbon materials.

A person skilled in the art will, however, be aware that, owing to the abovementioned probability considerations, a shorter milling time and a longer milling time are quite possible without going outside the scope of the present invention.

Factors essential to the present invention are not only the abovementioned probability but also the introduction of a minimum quantity of energy into the carbon nanoparticles being milled.

For the purposes of the present invention, such an energy input is in the range from 5 kJ/g_{carbon nanotubes} to 4000 kJ/g_{carbon nanotabes}. The energy input is preferably in the range from 500 kJ/g_{carbon nanotubes} to 2500 kJ/g_{carbon nanotabes}.

The energy input and the milling time are linked over a wide range via the milling power introduced. However, the energy input also depends on the milling apparatus as such.

However, the energy input should, for the same reasons given above in respect of the milling time, be within lower and in particular upper limits in order to prevent, in particular, formation of amorphous carbon.

In a particularly preferred embodiment of the present invention, a planetary mill is used as milling media mill and an energy of from 500 kJ/g_{carbon nanotubes} to 2500 kJ/g_{carbon nanotabes} is introduced over a milling time of from 4 to 8 hours.

This particularly preferred embodiment of the present invention is particularly advantageous because, firstly, the above probability of the carbon nanotubes being stressed at least once is maximized and, at the same time, the energy input is set so that amorphous carbon is obtained only in minimal amounts.

A very particularly preferred embodiment of the process of the invention is characterized in that carbon nanotubes are milled under a nitrogen atmosphere having a proportion of nitrogen of at least 90% by volume in a planetary mill for a period of from 4 to 8 hours with an energy input of from 500 kJ/g_{carbon nanotabes} to 2500kJ/g_{carbon nanotabes}.

The present invention further provides for the use of graphitic carbon materials which have been produced according to the invention and have pyridinic, pyrrolic and/or quaternary nitrogen groups at least on their surface in polymers, ceramics or metals as the constituent of a composite for improving the electrical and/or thermal conductivity and/or mechanical properties.

Use of the graphitic carbon materials which have been produced according to the invention and have pyridinic, pyrrolic and/or quaternary nitrogen groups at least on their surface for producing conductor tracks and conductive structures is likewise possible.

Other uses of the graphitic carbon materials which are produced according to the invention and have pyridinic, pyrrolic and/or quaternary nitrogen groups at least on their surface encompass use as storage medium, e.g. for hydrogen or lithium, in membranes, e.g. for the purification of gases, in the medical sector, e.g. as framework for growth control of cell tissue, in the diagnostic sector, e.g. as marker, and in chemical and physical analysis (e.g. in atomic force microscopes).

A preferred use according to the invention of the graphitic carbon materials which have been produced according to the invention and have pyridinic, pyrrolic and/or quaternary nitrogen groups at least on their surface is use thereof as catalysts in chemical reactions. The chemical reaction is preferably an electrochemical reaction. The chemical reaction particularly preferably comprises an electrochemical reduction of oxygen.

When used as catalyst in electrochemical reactions, use as electrode material in fuel cells and electrolysis cells is particularly preferred.

The above uses of the graphitic carbon materials which have been produced by the process of the invention and have pyridinic, pyrrolic and/or quaternary nitrogen groups are particularly advantageous because such an application becomes inexpensive, because it is simple, since the graphitic carbon materials which are obtained according to the invention and have pyridinic, pyrrolic and/or quaternary nitrogen groups no longer have to be obtained by reaction but can be obtained by purely mechanical methods under a nitrogen atmosphere and are nevertheless chemically modified with nitrogen groups.

The process of the invention and the preferred use of the carbon nanotubes milled under a nitrogen atmosphere as catalysts is illustrated below with the aid of examples, but the examples are not to be construed as a restriction of the inventive concept.

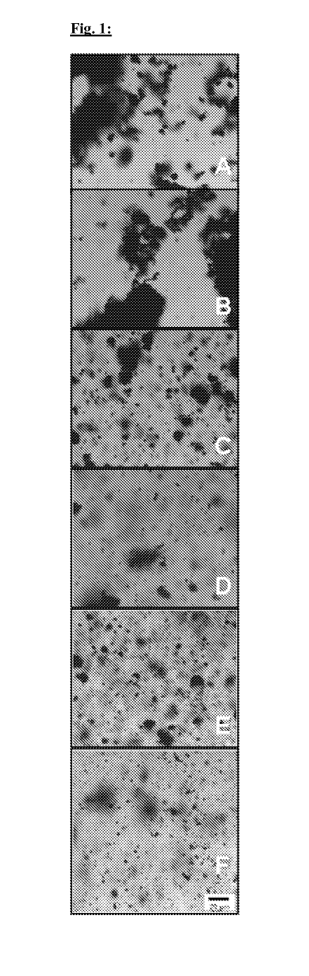

FIG. 1 shows the measurements as per Example 2 for samples which have been milled as described in Example 1 for two (A), six (B), ten (C) and thirty (D) minutes and also two (E) and sixteen (F) hours. The scale bar in the image of the sample after sixteen hours (F) likewise applies to the previous samples A to E.

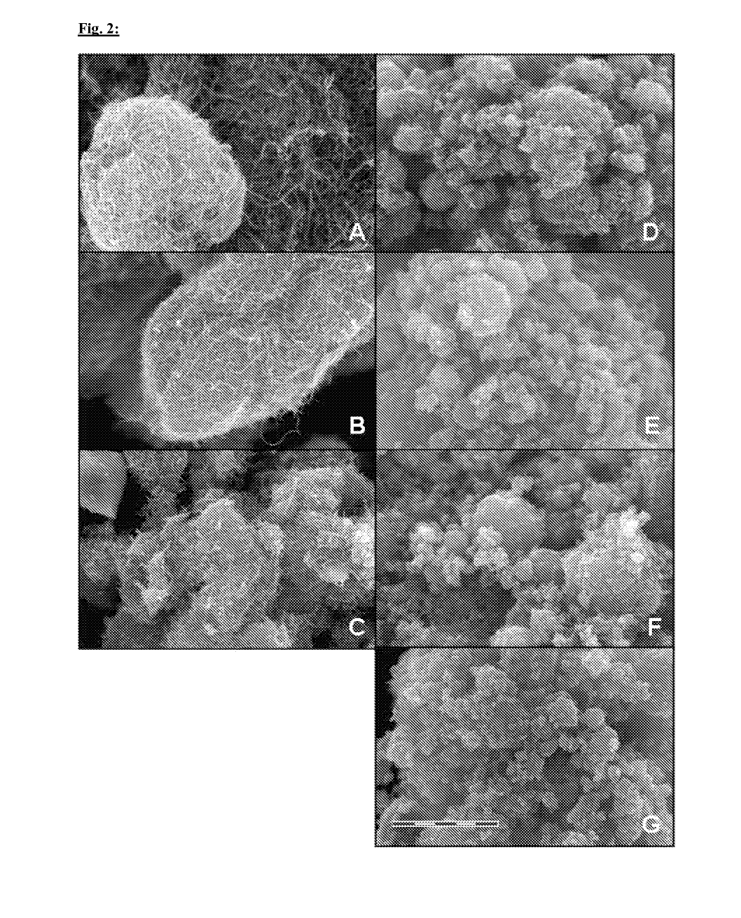

FIG. 2 shows the measurements as per Example 3 for samples at the beginning of the experiment (A) or which have been milled as described in Example 1 for two (B), fifteen (C) and thirty (D) minutes and also one (E), two (F) and sixteen (G) hours. The scale bar in the image of the sample after sixteen hours (G) corresponds to a length of 2 pm and likewise applies to the previous samples A to F.

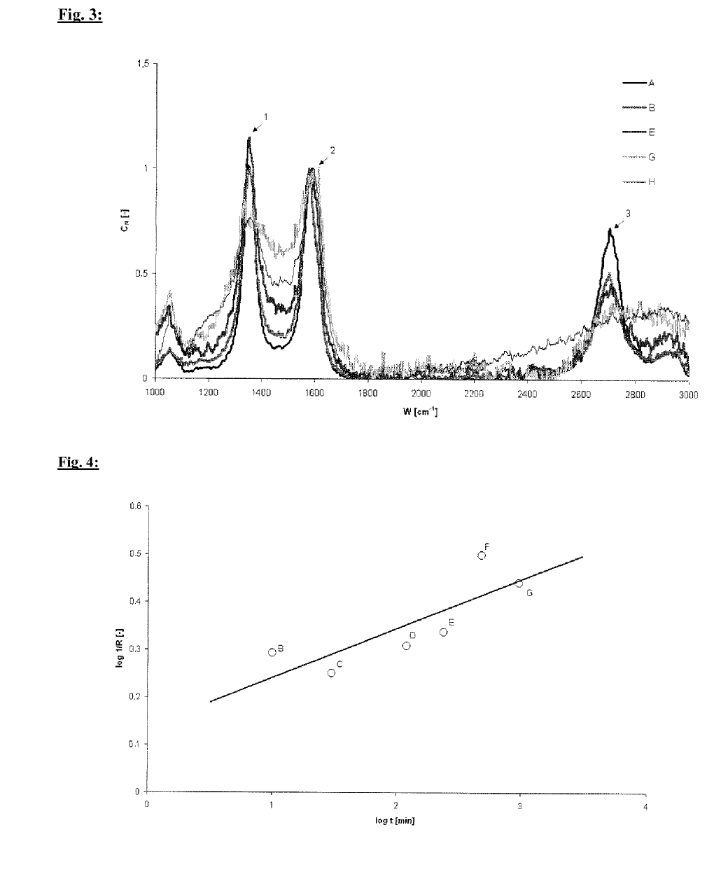

FIG. 3 shows measurement results as per Example 4, with only the measured values normalized to the graphite peak (2) (C_{N}) being shown against wave number (W) for measurements on unmilled carbon nanotubes (A), carbon nanotubes milled for 10 minutes (B), carbon nanotubes milled for four hours (E), carbon nanotubes milled for sixteen hours (G) and for carbon black (H). The FIGS. 1, 2, 3) denote the relevant measured peaks as per Example 4, which in the case of the samples A, B, E, G and H allow significant statements to be made about their defect density.

FIG. 4 shows a log-log plot of the defect density (1/R) against the milling time (t) in minutes for the measurements of the carbon nanotubes as described in Example 4 after 10 (B), 30 (C), 120 (D), 240 (E), 480 (F) and 960 (G) minutes.

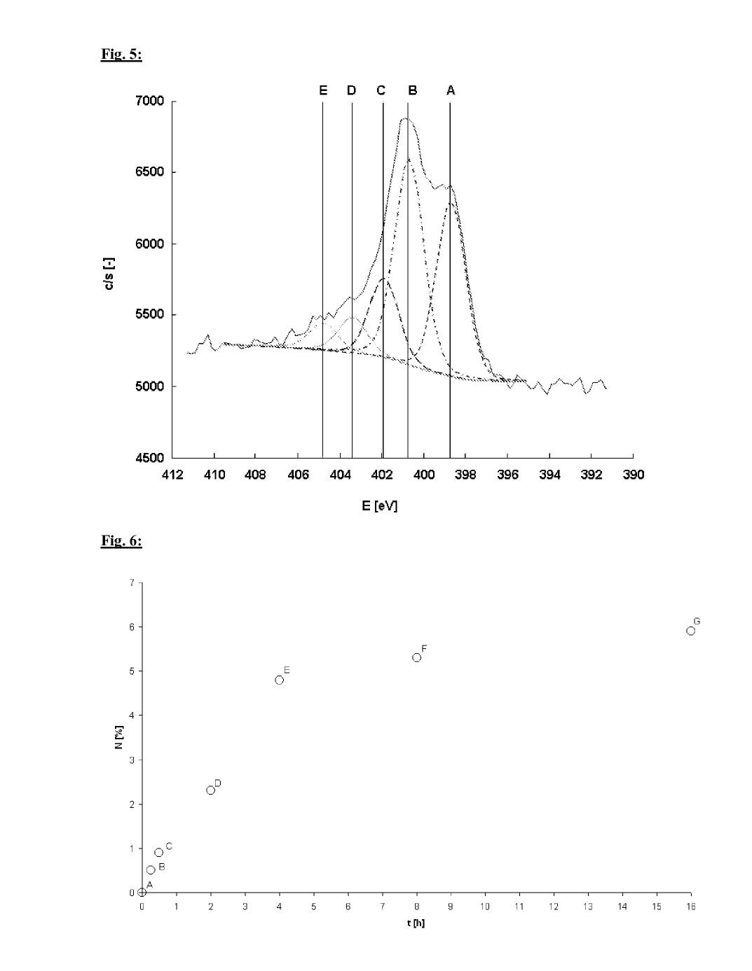

FIG. 5 shows the measurement result as per Example 5 after a milling time of eight hours. The actual measurement signal (c/s) is plotted against the binding energy (E) as a solid black line, and under the actual measurement signal idealized measurement signals for various possible types of bonding of nitrogen. Here, the idealized measurement signal of pyridinically bound nitrogen (A) is shown as a black line made up of short dashes having a maximum at a binding energy of 398.67 eV, the idealized measurement signal of pyn⁻olically bound nitrogen (B) is shown as a black dot-dash line having a maximum at a binding energy of 400.67 eV, the idealized measurement signal of quaternary nitrogen (C) is shown as a black line made up of long dashes having a maximum at a binding energy of 401.87 eV, the idealized measurement signal of nitrogen bound pyridinically as nitrogen oxide (D) is shown as a solid grey line having a maximum at a binding energy of 403.37 eV and the idealized measurement signal of nitrogen bound as nitrogen oxide (E) is shown as a grey line made up of long dashes having a maximum at a binding energy of 404.77 eV.

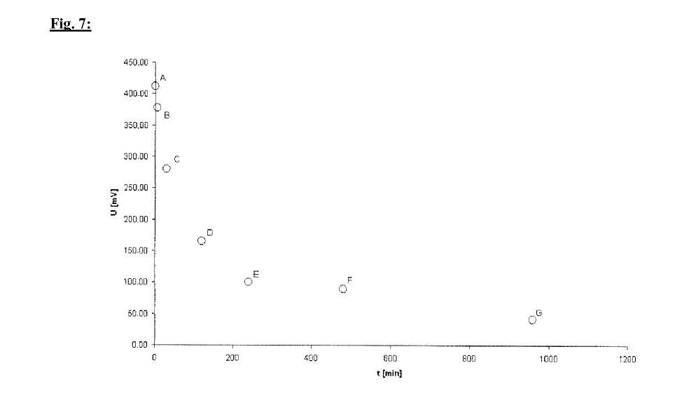

FIG. 6 shows the proportions of nitrogen on the surface of the carbon materials in atom % (N) determined as described in Example 5 plotted against the milling time (t) for the samples measured before commencement of the experiment (A), after fifteen (B) and thirty minutes (C) and also after two (D), four (E), eight (F) and sixteen hours (G).

FIG. 7 shows the reduction in the overvoltages (U) for the reduction of oxygen as a function of the milling time (t) by materials before commencement of the experiment (A), after six (B) and thirty (C) minutes, after two (D), four (E), eight (F) and sixteen (G) hours as per Example 6.

### EXAMPLES

### Example 1

### Process of the Invention

8 g of carbon nanotubes (BayTubes®C 150 P, from Bayer MaterialScience AG) were introduced into steel containers for the planetary mill model PM4 from Retsch GmbH in which 530 g of steel balls having a diameter of 2 mm are present. The introduction of the Baytubes and the closing of the containers took place in a glovebox which was continually flushed with pure nitrogen. Thus, an about 100% by volume nitrogen atmosphere prevailed in the interior of the milling chamber.

The planetary mill was operated for a time of sixteen hours, with samples being taken before commencement of the experiment, after one, two, six, ten, fifteen and thirty minutes and also after one, two, four, eight and sixteen hours and subjected to a confocal laser scanning microscopy (CLSM) examination as described in Example 2 and/or a scanning electron miscroscopic (SEM) examination as described in Example 3 and/or a Raman spectroscopic examination as described in Example 4 and/or an XPS (photoelectron spectroscopy) examination as described in Example 5 and/or were used as catalyst as described in Example 6.

### Example 2

### Confocal Laser Scanning Microscopy Examination

The samples after two (A), six (B), ten (C) and thirty (D) minutes and also after two (E) and sixteen (F) hours were examined under a confocal laser scanning microscope (CLSM) model TCS-NT (from Leica).

The results of the examinations are summarized in FIG. 1. It can be seen that the number of agglomerates of carbon nanotubes decreases with increasing milling time.

### Example 3

### Scanning Electron Microscopic (SEM) Examination

The samples before commencement of the experiment (A), after two (B), fifteen (C) and thirty (D) minutes and also after one (E), two (F) and sixteen (G) hours were examined under a scanning electron microscope (SEM) model S-FEG Sirion 100T (from FEI Company).

The results of the examinations are summarized in FIG. 2. It can be seen that the average length of the carbon nanotubes decreases with increasing milling time. Furthermore, the result of the examination as per Example 2 confirms that the average size of the agglomerates of carbon nanotubes decreases. Furthermore, it can be seen from the examinations after from one to sixteen (E to F) hours that after these times some amounts of amorphous carbon are present on the surface of the agglomerates. In particular, the still graphitic carbon materials obtained would no longer come under the generally customary definition of carbon nanotubes. In any case, only few structures which can clearly be identified as carbon nanotubes can be observed on the surface.

### Example 4

### Raman Spectroscopic Examination

The samples before commencement of the experiment (A), after ten (B) and thirty (C) minutes and also after two (D), four (E), eight (F) and sixteen (G) hours were examined using a Raman spectrometer model INDURAM (from Horiba Jobin Ivon). In addition, carbon black (H, XC72R procured from Vulcan) was examined in an analogous way.

Some of the results of the examination are summarized in FIG. 3, with only the samples before commencement of the experiment (A), after ten minutes (B), after four (E) and sixteen (G) hours and also carbon black (H) being shown for reasons of clarity.

In FIG. 3, it can be seen that the measured spectrum increasingly approaches that of carbon black (H). The milling process firstly increases the width at half height of the first defect peak (1) as a wave number of about 1350 cm⁻¹ and of the graphite peak (2) at a wave number of about 1580cm⁻¹. Secondly, a significant decrease in the intensity of the second defect peak (3) at a wave number of about 2700 cm⁻¹ can be seen.

In FIG. 4, the logarithm of the defect density (1/R) determined using the formula (I)

$$R = \frac{I ( 3 )}{I ( 1 )} \qquad ( I )$$

is plotted against the logarithm of the milling time in minutes. According to the formula (I), 1/R is the ratio of the measured value of the highest intensity of the graphite peak I(1) to the measured value of the highest intensity of the second defect peak I(3).

In FIG. 4, the respective values for the samples after ten (B), thirty (C) minutes and also after two (D), four (E), eight (F) and sixteen (G) hours are shown. The value before commencement of the experiment (t=0 min.) is not shown because the logarithm is indeterminate. The value for carbon black (H) is not shown because the second defect peak of a material which is already amorphous cannot be identified clearly.

It can be seen from FIG. 4 that there is a linear relationship between the logarithm of the milling time and the logarithm of the defect density, which makes it possible to optimize the milling time in respect of the resulting nonamorphous carbon nanotubes. It can also be seen therefrom that the milling time according to the invention is advantageous because complete conversion into the amorphous state has not yet occurred up to this point.

The fact that the defect density can be determined using the formula (I) is generally known (e.g. Vix-Guterl et al. J. Phys. Chem. B, 108 (2004) 19361).

### Example 5

### XPS Examination

The samples before commencement of the experiment (A), after eight (B) and sixteen (C) hours were examined using an XPS instrument (XPS) from ULVAC PHI, model VersaProbe. Monochromatic Al Kα radiation (1486.6 eV) was used for excitation.

The experimental results after eight hours (B) is shown in FIG. 5. The solid black line of the actual measurement signal (c/s) of the N1s line plotted against the binding energy (E) can be seen. The measurement signals of the different types of bonding of nitrogen possible for the assumed nitrogen bonds are drawn in under the measurement signal, so that the superposition of the measurement signal for different bonding states gives the real measurement signals very accurately.

In the measurement signal of nitrogen as depicted as a section in FIG. 5, it is possible to see 5 different bonding states: pyridinically bound nitrogen (A, black line made up of short dashes) at a binding energy of 398.67 eV, pyrrolically bound nitrogen (B, black dot-dash line) at a binding energy of 400.67 eV, quaternary nitrogen (C, black line made up of long dashes) at a binding energy of 401.87 eV, nitrogen bound pyridinically as nitrogen oxide (D, solid grey line) at a binding energy of 403.37 eV and nitrogen present as nitrogen oxide (E, grey line made up of long dashes) at a binding energy of 404.77 eV.

The proportion of nitrogen in atom % of the measured surface was determined for the measured values from the samples before commencement of the experiment (A), after fifteen (B) and thirty minutes (C) and also after two (D), four (E), eight (F) and sixteen hours (G) from the data in FIG. 5 by integration and conversion of the areas under the lines A to E.

The results are summarized in FIG. 6. While no nitrogen could be found at the commencement of the experiment (A), about 6 atom % of nitrogen were detected on the surface of the carbon materials after sixteen (G) hours.

### Example 6

### Use of the Carbon Nanotubes Milled Under a Nitrogen Atmosphere

To examine the electrochemical, catalytic properties of the graphitic carbon materials having pyridinic, pyrrolic and/or quaternary nitrogen groups at least on their surface which are obtained as described in Example 1, suspensions of the carbon materials obtained from Example 1 were firstly produced by in each case adding 33.3 mg of carbon material to 100 ml of acetone in a conical flask and then treating the mixture in an ultrasonic bath for 30 minutes in each case in order to obtain a very homogeneous suspension.

The suspension obtained in this way was applied to a working electrode in the form of a rotating annular disc electrode (electrode material: Glassy-Carbon, from Metrom) by applying 5 μl of this suspension four times in sequence to this working electrode and then in each case evaporating the acetone under ambient conditions (23° C., 1013 hPa). A layer of a fluorinated hydrocarbon (Nafion®, from DuPont) was applied on top of the carbon materials now present on the working electrode by applying 5 μl of a suspension comprising 0.26 mg/ml of fluorinated hydrocarbon in isopropanol and subsequently evaporating the isopropanol again under ambient conditions (23° C., 1013 hPa).

The working electrode was, after the above preparation steps, in each case hung into a 5% strength by weight hydrogen chloride solution which was maintained at 60° C. and had been saturated with oxygen by direct introduction of gas.

The measurement was then carried out in a 3-electrode arrangement using the working electrode, a silver/silver chloride reference electrode and a platinum counter electrode. The potential between the working electrode and reference electrode was set via an electric circuit and control (potentiostat as voltage source). At the same time, it is ensured by means of the voltage source that the current flows only through the working electrode and the counterelectrode and the reference electrode remains without current.

A measurement potential to the working electrode was in each case set by means of the adjustable voltage source starting from a potential of 0.8 V relative to the abovementioned silver/silver chloride reference electrode at a rate of 20 mV/s to a potential of −0.2 V relative to the abovementioned silver/silver chloride reference electrode.

The resulting current was measured as a function of the potentials. The above procedure was repeated eight times in each case.

The real overvoltage which indicated the further voltage (further potential) which was required under real conditions in order to bring about actual reduction of oxygen could be determined from the measured data for current as a function of applied potential using the generally known, theroretically necessary potential for the reduction of oxygen (+0.401 V).

Since the graphitic carbon materials which have been produced according to the invention and comprise pyridinic, pyrrolic and/or quaternary nitrogen groups at least on their surface are catalytically active compounds for such a reduction reaction, this overvoltage which can, for instance, be attributed to kinetic limitation of the reaction was reduced.

The potentials which had to be applied at a current of 200 μA for materials before commencement of the experiment (A), after six (B) and thirty (C) minutes and after two (D), four (E), eight (F) and sixteen (G) hours were compared.

In FIG. 7, the overvoltages (U) found are shown as a function of the milling time (t).

It can be seen that the overvoltage decreases exponentially as a function of the milling time. This leads to the advantageous use according to the invention of the resulting graphitic carbon materials comprising pyridinic, pyrrolic and/or quaternary nitrogen groups at least on their surface for the electrochemical reduction of oxygen.

In particular, it could in this way be established that a milling time of up to eight hours represents a certain optimum for this, since a further doubling of the milling time to sixteen hours did not lead to such a large further reduction in the overvoltage to make this seem worthwhile.

1. Process for producing graphitic carbon materials comprising pyridinic, pyrrolic and/or quaternary nitrogen groups at least on their surface starting out from carbon nanotubes, wherein carbon nanotubes are milled under a nitrogen atmosphere.

2. Process according to claim 1, wherein the nitrogen atmosphere has a proportion of nitrogen of at least 90% by volume, preferably at least 99% by volume.

3. Process according to claim 1, wherein the milling is carried out in a milling media mill.

4. Process according to claim 3, wherein the milling is carried out in a planetary mill.

5. Process according to claim 1, wherein the milling is carried out for a period of from one minute to sixteen hour.

6. Process according to claim 1, wherein an amount of energy of from 5 kJ/g_{carbon nanotubes} to 4000 kJ/g_{carbon nanotubes} is introduced during milling.

7. A method for carrying out chemical reactions, which comprises carrying out such chemical reactions in the presence of a graphitic carbon material of claim 1 as catalyst.

8. The method of claim 7, wherein the chemical reaction is an electrochemical reaction.

9. The method of claim 8, wherein the electrochemical reaction comprises a reduction of oxygen.

10. Method of claim 8 wherein said graphitic carbon material is present as electrode material in fuel cells and electrolysis cells.

11. Process of claim 5, wherein the milling is carried out for a period of from four to eight hours.

12. Process of claim 6, wherein said amount of energy is in the range of from 500 kJ/g_{carbon nanotubes} to 2500 kJ/g_{carbon nanotubes}.
